(19) United States
(12) Patent Application Publication
(10) Pub. No.: US 2018/0325813 A1
(43) Pub. Date: Nov. 15, 2018

(54) ORAL SOLUTION OF ACE INHIBITORS

(71) Applicants: Jayanta Kumar MANDAL, Satelite, Ahmedabad (IN); FTF Pharma Private Limited, Taluka: Sanand Dist.: Ahmedabad (IN)

(72) Inventors: Jayanta Kumar Mandal, Satelite, Ahmedabad (IN); Henil Alpeshbhai PATEL, Ghatlodia, Ahmedabad Gujarat (IN); P. KANTEEPAN, Tamil Nadu (IN)

(73) Assignee: FTF Pharma Private Limited, Taluka: Sanand Dist.: Ahmedabad (IN)

(21) Appl. No.: 15/774,073

(22) PCT Filed: Oct. 27, 2016
(86) PCT No.: PCT/IB2016/056446

§ 371 Date: May 7, 2018

(30) Foreign Application Priority Data
Nov. 7, 2015 (IN) 4253/MUM/2015

(51) Int. Cl. A61K 9/00; A61K 31/401; A61K 47/26; A61K 47/14; A61K 9/08; A61K 9/10

(52) U.S. Cl. CPC A61K 9/0053; A61K 31/401; A61K 9/10; A61K 47/14; A61K 9/08; A61K 47/26

(57) ABSTRACT

The present invention provides pharmaceutical oral solution composition of ACE inhibitor. The oral solution composition comprises of ACE inhibitor with 5-9 pH and other pharmaceutical excipients having improved stability and palatability.

### FIELD OF INVENTION

The present invention relates to the field of oral pharmaceutical composition of ACE inhibitors. In particular, present invention relates to oral solution composition comprises of ACE inhibitor with pH 5-9 with improved stability and improved palatability.

### BACKGROUND OF INVENTION

Since their introduction, angiotensin-converting enzyme inhibitors (ACEIs) have come to be regarded as a major advance of hypertension.

Angiotensin-converting enzyme inhibitors were developed from a serendipitous discovery that a brazilian snake venom contained a bradykinin potentiating factor with vasodilating properties. The physiological significance of this was established by demonstrating inhibition of ACE by the peptide mixture from the snake venom. The first clinically usable ACEIs, captopril, was synthesized in 1975, followed by enalapril, lisinopril, benazepril, fosinopril, ramipril, quinapril.

Angiotensin-converting enzyme (ACE) inhibitors have been demonstrated to reduce morbidity and mortality rates in patients with heart failure with left ventricular systolic dysfunction. Angiotensin Converting Enzyme plays a central role in the control of peptide hormones that regulate blood pressure and thereby angiotensin-converting enzyme inhibitors are effective antihypertensive agents.

Angiotensin converting enzyme inhibitors are generally very difficult to formulate into dosage forms as certain angiotensin converting enzyme inhibitors upon contact with commonly used pharmaceutical excipients are prone to various types of degradation such as cyclization via internal nucleophilic attack to form substituted diketopiperazines, hydrolysis of the side-chain ester group and oxidation to form products having often unwanted coloration.

U.S. Pat. No. 4,830,853 disclose that oxidation and color stability of certain ACE inhibitors is optimized when they are formulated with a stabilizer and at least one lubricant and/or excipient.

U.S. Pat. No. 4,743,450 disclose stable compositions containing ACE inhibitors with metal-containing stabilizer such as magnesium carbonate and saccharide. As magnesium carbonate is bulky in nature and has poor compressibility, moldability, and flowability which causes difficulties during tablet formulation.

U.S. Pat. No. 6,462,022 disclose pharmaceutical tablet formulation comprising of lisinopril and large particle sized dibasic calcium phosphate dihydrate with increased shelf life of tablets.

CN 103006612 discloses sustained-release delayed tablet of lisinopril.

CN 104147588 discloses stable lisinopril tablets and method of its preparation.

CN 104523629 lisinopril freeze-dried tablet composition method of its preparation.

Although each of the above patents represent an attempt to formulate solid dosage form with different excipients to overcome the instability and degradation related problems associated with ACE inhibitors containing compositions, there still exists a need for ACE inhibitor containing formulation having improved stability and improved palatability. To this end, the present invention is directed to oral solution formulation of ACE inhibitors with preservative exhibiting improved stability and palatability.

### OBJECT OF INVENTION

The primary object of present invention is to provide oral pharmaceutical solution of ACE inhibitors with pH 5-9 to overcome degradation to diketopiperazines.

Another object of present invention is to provide oral pharmaceutical solution of ACE inhibitors with improved stability towards hydrolysis and oxidation.

Another object of present invention is to provide a cost effective and highly patient compliance oral solution of ACE inhibitors.

It is yet another object of present invention is to provide a process for preparation of stabilized oral pharmaceutical solution of ACE inhibitors.

### SUMMARY OF INVENTION

The present invention relates to an oral solution of ACE inhibitors with preservative to overcome degradation and having improved stability and palatability.

According to one aspect of present invention, there is provided an oral pharmaceutical solution of ACE inhibitors with preservative which protect lisinopril from degradation into diketopiperazines, side chain ester hydrolysis or oxidation.

Another aspect of present invention relates to oral solution of ACE inhibitors comprises of an active ingredient with a preservative and other pharmaceutically acceptable excipients such as vehicle, sweetener, buffering agent and flavouring agent.

### DETAIL DESCRIPTION OF PRESENT INVENTION

Angiotensin-converting enzyme inhibitors block the activity of an enzyme that causes blood vessels to constrict and as a result blood vessels relaxes and widens and makes the flow of blood easier through the vessels which reduces blood pressure. Preventing blood vessels from narrowing helps to improve blood flow and reduces the backup of blood in the heart and lungs which further decreases the pressure that the heart's left ventricle must pump against.

As there are certain angiotensin-converting enzyme inhibitors which upon contact with some of the pharmaceutical excipients undergo various types of degradation having often unwanted coloration are as following:

1) Cyclization via internal nucleophilic attack to form substituted diketopiperazines,
2) Hydrolysis of the side-chain ester group,

### 3) Oxidation.

In an embodiment, the active pharmaceutical ingredient for oral pharmaceutical solution dosage form is selected from ACE inhibitors such as captopril, enalapril, ramipril, quinapril, perindopril, lisinopril, benazepril, fosinopril, imidapril, trandolapril, cilazapril.

In an embodiment, the present invention relates to one of the ACE inhibitor, lisinopril dihydrate.

Chemically, lisinopril is (2S)-1-[(2S)-6-amino-2-[[(1S)-1-carboxy-3-phenylpropyl]amino] hexanoyl]pyrrolidone-2-carboxylic acid. Lisinopril has following chemical structure:

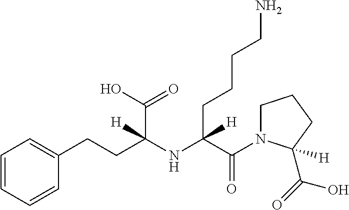 [SMILES: NCCCC[C@@H](N[C@H](CCc1ccccc1)C(=O)O)C(=O)N1CCC[C@H]1C(=O)O]

Lisinopril upon contact with some of the pharmaceutical excipients converted into cyclized degradation product, lisinopril diketopiperazine having following structure:

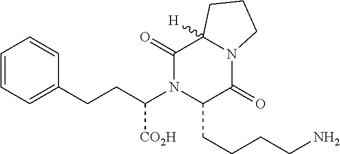 [SMILES: NCCCC[C@H]1C(=O)N2CCCC2C(=O)N1[C@@H](CCc1ccccc1)C(=O)O]

Further, lisinopril also get degraded by hydrolysis of side chain ester group or oxidation. It was surprisingly found that the combination of lisinopril and preservative in solution form results in enhanced stability of ACE-inhibitor, lisinopril toward cyclization, hydrolysis and oxidation.

Aspects of present invention relates to oral pharmaceutical solution of lisinopril dihydydrate comprising of active ingredient lisinopril dihydydrate with preservative and other pharmaceutically acceptable excipients such as vehicle, sweetener, buffering agent and flavouring agent.

Vehicles referred in present invention are mainly liquid bases which carry drugs and other excipients in dissolved or dispersed state and can be selected from either aqueous vehicles or oily vehicles. Aqueous vehicles can be selected from but not limited to purified water, hydro-alcoholic, polyhydric alcohols and buffers, while oily vehicles can be selected from vegetable oils, mineral oils, organic oily bases or emulsified bases.

By preservatives in the present invention, it refers to compounds which are used to control the microbial bioburden of the formulation having broad spectrum of antimicrobial activity, must be chemically and physically stable over the shelf-life of the product and have low toxicity, which can be selected from group but not limited to alcohol, benzyl alcohol, chlorbutol, chlorocresol, alkyl esters of paraben, phenol, phenyl ethanol, sodium benzoate, antimicrobial solvents like propylene glycol, chloroform.

Sweetener can be selected from but not limited to sucrose, sucralose, liquid glucose, liquid maltitol, glycerol, sorbitol, saccharin sodium and aspartame to impart sweetness to the formulation.

Buffering agents are mainly selected based on their suitability for use in oral liquids, the stability of the formulation in the presence of the buffer and compatibility of the buffer with the product container. Buffering agent can be selected from but not limited to sodium acetate, sodium hydroxide, sodium citrate, sodium phosphate and disodium phosphate.

By flavoring agents as referred to the present invention, are mainly use to increase the palatability and enhance the aesthetic qualities of the liquid formulation. Flavoring agent can be selected but not limited to oil based flavoring agent such as essential oils including peppermint oil, orange oil, and lemon oil etc or can be selected from fruit flavors.

### EXAMPLES

The present invention can be described by way of example only. It is to be recognized that modifications falling within the scope and spirit of the description or claims, which would be obvious to a person skilled in the art based upon the disclosure herein, are also considered to be included within the scope of this disclosure.

For the composition of lisinopril 1 mg/ml-4 mg/ml, drug and excipients with its range are shown below in table:

| | | Formula | Formula |
|---|---|---|---|
| Sr. No | Name of Ingredient | (mg/ml) | (% W/V) |
| 1 | Lisinopril dihydrate | 2-6 | 0.2 to 0.6% |
| 2 | Liquid maltitol | 150-170 | 15-17% |
| 3 | Sucralose (optional) | 0-2 | 0-0.02% |
| 4 | Methyl paraben | 1-5 | 0.1 to 0.5% |
| 5 | Ethyl paraben | 0-0.5 | 0-0.05% |
| 6 | Strawberry flavor | 0-0.5 | 0-0.05% |
| 7 | Sodium dihydrogen | 1-3 | 0.1 to 0.3% |
| | phosphate | | |
| 8 | Sodium dihydrogen | 2-5 | 0.2 to 0.5% |
| | phosphate | | |
| 10 | Purified water | QS | Up to 100% |

The oral pharmaceutical solution of above composition is prepared by following method:—
a) Heat 50% of total quantity of purified water at 75° C.−80° C. and maintain temperature up to 80° C.
b) Add preservative and stir it till clear solution obtained.
c) Cool above prepared solution of step b) at room temperature.
d) Take 10% of total quantity of water and add buffering agent to it.
e) Add solution of step d) to the solution of step c).
f) Add lisinopril dihydrate to the solution of step e) with continuous stirring.
g) Add sweetener (liquid maltitol/sucralose) to the solution of step f) with continuous stirring.
h) Add flavoring agent (strawberry flavor) the solution of step g) with continuous stirring.
i) Make up the volume of solution of step h) with remaining quantity of water.

### Example 1

For the composition of lisinopril 4 mg/ml, using less excepients is shown below in table:

| | | Formula |
|---|---|---|
| Sr. No | Name of Ingredient | (mg/ml) |
| 1 | Lisinopril dihydrate | 4 |
| 2 | Sucralose | 1 |
| 3 | Methyl paraben | 1.6 |
| 4 | Ethyl paraben | 0.2 |
| 5 | Strawberry flavor | 0.1 |
| 6 | NaOH | QS |
| 7 | Purified Water | QS |

The oral pharmaceutical solution of above composition is prepared by following method:—
a) 80% of purified water taken in stainless steel vessel, heated to 80-85° and under continuous stirring methyl paraben, ethyl paraben were added and allow to cool at room temperature.
b) To above clear solution weighed quantity of API were added slowly and stirred for homogeneity.
c) To above prepared solution sucralose and strawberry flavor added and stirred for homogeneity.
d) To prepare NaoH solution and added to step 3 dropwise to adjust targeted pH (5.5-6.5) and more closely to 6.0
e) Volume make up using purified water.
f) Filter above solution using 10 micron Polypropylene filter

### Example 2

For the composition of lisinopril 1 mg/ml, using less excepients is shown below in table:

| | | Formula |
|---|---|---|
| Sr. No | Name of Ingredient | (mg/ml) |
| 1 | Lisinopril dihydrate | 1 |
| 2 | Sucralose | 1 |
| 3 | Methyl paraben | 1.6 |
| 4 | Ethyl paraben | 0.2 |
| 5 | Strawberry flavor | 0.1 |
| 6 | NaOH | QS |
| 7 | Purified Water | QS |

The oral pharmaceutical solution of above composition is prepared by following the method of example 1.

Using the above composition of example 1 and 2, suspension can also be prepared by following method:—
a) Take 70/Purified water of total batch size, heated to 80-85° and add Methylparaben and Ethylparaben. Allow to cool at room temperature.
b) Add and dissolve liquid maltitol or in step 1.0 under stirring.
c) Add and dissolve API (active pharmaceutical ingredient) in step 2.0 under stirring.
d) Add and dissolve sucralose in step 4.0 under stirring.
e) To prepare NaoH solution and added to step 3 dropwise to adjust pH between 4-8 and more closely to 6.0
f) Add and mixed flavouring agent in step 5.0 under stirring.
g) Finally volume makes up by Purified water and mix properly under stirring.

We claim:

1) A pharmaceutical oral solution of ACE inhibitor stable at pH 5-9 comprises stable buffer system and pH modifier having improved stability and improved palatability.

2) A pharmaceutical oral solution of ACE inhibitor as claimed in claim 1, wherein ACE inhibitor can be selected from captopril, enalapril, ramipril, quinapril, perindopril, lisinopril, benazepril, fosinopril, imidapril, trandolapril, cilazapril or a pharmaceutically acceptable salt thereof.

3) A pharmaceutical oral solution of ACE inhibitor as claimed in claim 2, wherein ACE inhibitor is lisinopril or a pharmaceutically acceptable salt thereof.

4) A pharmaceutical oral solution of ACE inhibitor derivative as claimed in claim 1 to 3, wherein dihydropyridine derivative is lisinopril dihydrate.

5) A pharmaceutical oral solution as claimed in claim 1, wherein other pharmaceutically accepted excipients are selected from vehicle, sweetener, buffering agent and flavoring agent.

6) A process for preparation of a pharmaceutical oral solution of lisinopril dihydrate comprises of following steps:
a) Heating of 50% of total quantity of purified water at 75° C.-80° C. and maintain temperature up to 80° C.
b) Addition preservative and stirring it till clear solution obtained.
c) Cooling of above prepared solution of step b) at room temperature.
d) Addition of buffering agent to 10% of total quantity of water.
e) Addition of solution of step d) to the solution of step c).
f) Addition of lisinopril dihydrate to the solution of step e) with continuous stirring.
g) Addition of sweetener (liquid maltitol/sucralose) to the solution of step f) with continuous stirring.
h) Addition of flavoring agent (strawberry flavor) to the solution of step g) with continuous stirring.
i) Adjustment of volume of solution of step h) with remaining quantity of purified water.

7) A pharmaceutical oral solution of lisinopril 4 mg/ml, comprises lisinopril dehydrate, sucralose, methyl paraben, ethyl paraben, flavoring agent, pH adjusting agent and purified water.

8) A pharmaceutical oral solution of lisinopril 4 mg/ml, as claimed in claim 7 comprises Lisinopril dihydrate prepared by using following method:—
a) 80% of purified water taken in stainless steel vessel, heated to 80-85° and under continuous stirring methyl paraben, ethyl paraben were added and allow to cool at room temperature.
b) To above clear solution weighed quantity of API were added slowly and stirred for homogeneity.
c) To above prepared solution sucralose and strawberry flavor added and stirred for homogeneity.
d) To prepare NaoH solution and added to step 3 dropwise to adjust targeted pH (5.5-6.5) and more closely to 6.0
e) Volume make up using purified water.
f) Filter above solution using 10 micron Polypropylene filter

9) A pharmaceutical oral suspension of lisinopril and other pharmaceutically accepted excipients are selected from vehicle, sweetener, buffering agent and flavoring agent.

10) A pharmaceutical oral suspension of lisinopril as claimed in claim 6, wherein suspension is prepared by following method:—
a) Take 70%/Purified water of total batch size, heated to 80-85° and add Methylparaben and Ethylparaben. Allow to cool at room temperature.
b) Add and dissolve liquid maltitol or in step 1.0 under stirring.
c) Add and dissolve API (active pharmaceutical ingredient) in step 2.0 under stirring.
d) Add and dissolve sucralose in step 4.0 under stirring.
e) Prepare NaoH solution and added to step 3 dropwise to adjust pH 6.0
f) Add and mixed flavouring agent in step 5.0 under stirring.
g) Finally volume makes up by purified water and mix properly under stirring.
